(19) United States
(12) Patent Application Publication
(10) Pub. No.: US 2008/0085294 A1
(43) Pub. Date: Apr. 10, 2008

(54) Apparatuses and methods to treat atherosclerotic plaques

(76) Inventors: Toby Freyman, Waltham, MA (US); Robert Herrmann, Boston, MA (US); Wendy Naimark, Boston, MA (US); Maria Palasis, Wellesley, MA (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 11/538,683

(22) Filed: Oct. 4, 2006

(51) Int. Cl. A61F 2/00

(52) U.S. Cl. 424/423

(57) ABSTRACT

This invention comprises an apparatus for treating an atherosclerotic plaque in a coronary artery of a mammal by placing at or proximate to an entrance to the coronary artery and upstream of the vulnerable plaque, and an effective amount of a therapeutic agent for the treatment of the plaque. This invention comprises delivering a deposition of a therapeutic drug high in the coronary arterial tree for treatment of downstream vulnerable plaques. The invention also comprises slow release formulation and delivery of an apparatus that is totally degradable or removed and/or replaced.

### BACKGROUND

The disclosed inventions relate generally to medical devices and methods, and more particularly to methods, apparatuses and formulations directed to the treatment of vulnerable plaques in coronary arteries.

Cardiovascular disease is one of the leading causes of deaths worldwide. Traditionally, cardiovascular disease was thought to originate from severe blockages created by atherosclerosis, the progressive accumulation of non-vulnerable plaques in the coronary arteries. This constriction or narrowing of the affected vessel could ultimately lead to angina, and eventually coronary occlusion, sudden cardiac death, and/or thrombotic stroke.

Recent studies have lead to a shift in understanding of atherosclerosis. Scientists now believe that at least some coronary diseases involve an inflammatory process, in which inflammation causes atherosclerotic plaques to rupture. This inflamed plaque is known as atherosclerotic vulnerable plaque (vulnerable plaque). Recent studies have suggested that plaque rupture may trigger 60% to 70% of fatal myocardial infarctions. Of those, 25% to 30% are triggered by plaque erosion or ulceration.

Studies into the composition of vulnerable plaque suggest the presence of inflammatory cells. A large lipid core with associated inflammatory cells is the most powerful predictor of ulceration and/or imminent plaque rupture. For example, in plaque erosion, the endothelium beneath the thrombus is replaced by or interspersed with inflammatory cells.

Another feature of a vulnerable plaque is breakdown of connective tissues. There is a body of evidence indicating that matrix metalloproteases (MMPs) are important in the uncontrolled breakdown of connective tissue, including proteoglycan and collagen, leading to resorption of the extracellular matrix. Normally MMPs are tightly regulated at the level of their synthesis as well as at their level of extracellular activity. A variety of extracellular stimuli, including cytokines, cell-to-cell, and cell-to-matrix interactions can induce MMP expression. Of particular relevance to atherosclerotic pathology is an increase of expression and activity of MMPs have been noted in vulnerable plaques regions (Galis et al. (1994) J. Clin. Invest., 94, 2493-2503).

Vulnerable atherosclerotic plaques are often undetectable using conventional techniques such as angiography. Indeed, the majority of these vulnerable plaques that lead to infarction occur in coronary arteries that appeared normal or only mildly stenotic on angiograms performed prior to the infarction. However, if vulnerable plaques are identified the treatment options are limited. Current treatments tend to be general in nature. For example, low cholesterol diets are often recommended to lower serum cholesterol (i.e. cholesterol in the blood). Other approaches utilize systemic anti-inflammatory drugs such as aspirin and non-steroidal drugs to reduce inflammation and thrombosis. However, it is believed that if vulnerable atherosclerotic plaque can be reliably detected, localized treatments may be developed to specifically address the problems.

One proposed approach to treating vulnerable plaques includes systemic delivery of a therapeutic agent. This approach entails undesirable side effects of the therapeutic agent and large quantities of agent required to deliver a therapeutically effective amount to the vulnerable plaque. Another proposed approach involves highly localized delivery of a therapeutic agent by eluting a drug from a stent placed in the lumen of the artery directly on the plaque. Drawbacks to this approach include the need to place a stent on the site of each vulnerable plaque, with attendant trauma and risk of stenosis and the jacketing of the artery with stents, which can be problematic if and when subsequent angioplasty/stenting procedures are needed. Further, it is desirable to treat patients who are susceptible to development of vulnerable plaques before a specific plaque develops or is identified. It is also desirable to treat vulnerable plaques that may develop further down in the coronary artery branches, where the artery lumen is smaller and more difficult to access with a stent.

Recently Wang, et al. (2004) Circulation 110, 278-284, published a geographical distribution of occlusive thromboses throughout the coronary tree. Wang, et al. demonstrated that such occlusions are clustered within the proximal portions of the major coronary arteries. Specifically, the authors reported that the spatial distribution of coronary thromboses causing ST segment elevation myocardial infarctions (STEMIs) are caused by unstable plaque erosions or ruptures focused in the large coronary arteries rather than within the smaller branches downstream of the larger coronary arteries. The study reported that acute STEMIs are highly clustered within the proximal portions of large epicardial arteries. These hot spots trend toward the proximal vessel, especially in the left anterior descending (LAD) artery. Locations where there are a high probability of vulnerable plaques are shown in FIG. 1. Because of their relative confinement to these segments (50% of LAD thromboses occurred within the first 25 mm of the vessel), therapeutic approaches should be designed to treat unstable plaques in these locations.

### SUMMARY OF THE INVENTION

In accordance with one embodiment of the invention, an apparatus for treating an atherosclerotic plaque in a coronary artery of a mammal is placed at or proximate to an entrance to the coronary artery and upstream of the vulnerable plaque, and releases an effective amount of a therapeutic agent for the treatment of the plaque. The apparatus includes some structure that enables it to serve as a reservoir for the therapeutic agent, and to maintain its location in the desired location in the ostium or artery.

The structure is preferably biodegradable, so that the device eventually is absent from the ostium or artery, but may also be partially or completely non-biodegradable and thus permanent. The reservoir functionality may be achieved by incorporating the therapeutic agent into the constituent material of the device to be eluted from the material (whether or not as part of the biodegradation of the material), by forming a cavity in the device from which the agent can be released, and/or by nanostructures on or in the apparatus.

The device may be configured as an annular ring, as a cylindrical stent, or in any other suitable shape. Retention of the device in the desired location can be achieved in many ways. It may be achieved by incorporating some retention mechanism, such as mechanical fastener (prong, spike, hook), or through adhesion (forming the device of, or coating with, a sticky/tacky material, and/or applying an adhesive). It may be accomplished simply by the geometry of the device, conforming the device more or less closely to the shape of the ostium or artery wall and relying on friction and/or the pressure gradient of the blood flowing through the artery and/or the device to hold the device in place. The device may be formed externally to the body and then inserted into the desired location, or may be formed in situ.

### BRIEF DESCRIPTION OF THE DRAWINGS

FIG. 1 shows a heart, right and left coronary arteries, and possible sites of vulnerable plaques.

FIG. 2 is a cross sectional view of the aortic arch, showing the ostia and portions of the left and right coronary arteries.

FIG. 3 is an enlarged cross-sectional view of a portion of FIG. 2, showing the left coronary artery and associated ostium and an exemplary device shown schematically.

FIG. 4 is a schematic illustration of an the device shown in FIG. 3.

FIGS. 5A and 5B illustrate a device according to a first embodiment, in a perspective view and in a cross-sectional view disposed in the entrance to a coronary artery.

FIGS. 5C and 5D illustrate alternative attachment mechanisms for securing the device according to the first embodiment to the arterial wall.

FIG. 5E illustrates an alternative embodiment with non-biodegradable core or scaffold.

FIG. 5F illustrates an alternative embodiment that includes a cavity to contain a therapeutic agent.

FIG. 5G illustrates an alternative embodiment in which the device has a tapered, annular shape.

FIGS. 6A and 6B illustrate a device according to a second embodiment disposed in the entrance to a coronary artery.

FIGS. 6C and 6D illustrate alternative attachment mechanisms for securing the device of FIGS. 6A and 6B to the arterial wall.

FIGS. 7A and 7B illustrate a mesh device according to a third embodiment disposed in the entrance to a coronary artery.

FIGS. 8A and 8B illustrate a tubular stent device according to a fourth embodiment disposed in the entrance to a coronary artery.

FIG. 9 illustrates a device according to a fifth embodiment disposed in the interior perimeter of the coronary artery.

FIG. 10 illustrates a plug device according to a sixth embodiment disposed in the entrance to a coronary artery.

FIG. 11 illustrates a device according to a seventh embodiment formed in situ in a coronary artery.

FIGS. 12A and 12B illustrate a device according to an eighth embodiment assembled in situ in a coronary artery from preformed sheets.

FIGS. 13A and 13B illustrate a device according to a ninth embodiment that includes an absorption inhibitor layer.

FIGS. 14A and 14B illustrate a device according to a tenth embodiment in which an external energy source is utilized to release therapeutic agent from a reservoir.

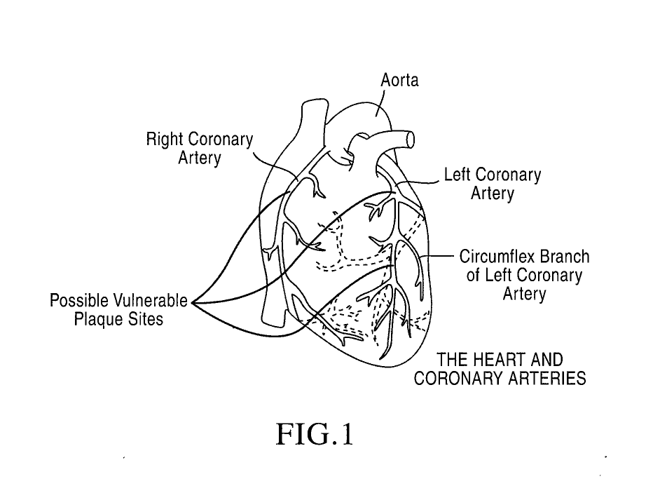

### DETAILED DESCRIPTION

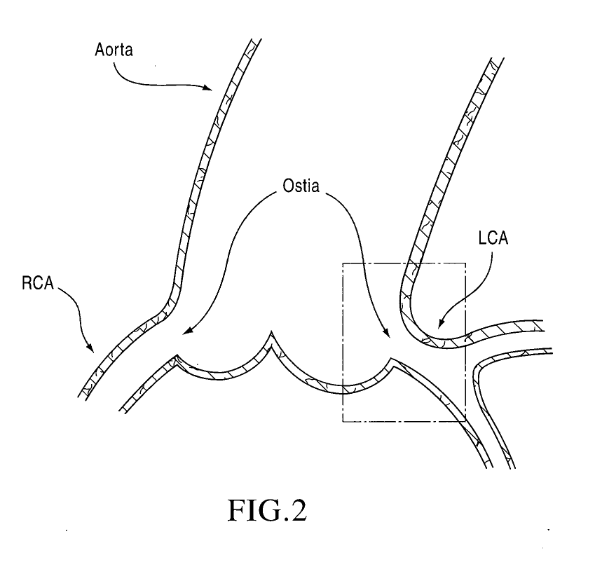

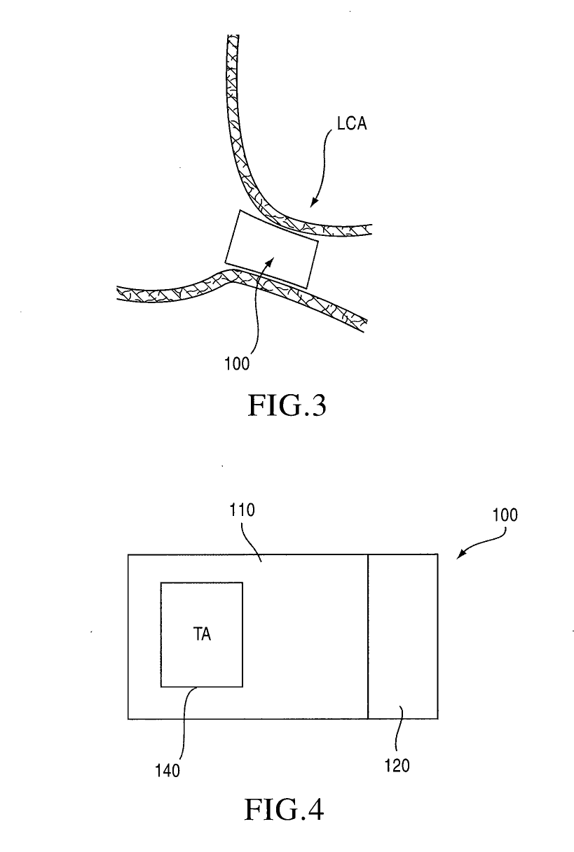

The aortic arch and the ostia and entrances to the left and right coronary arteries are shown in FIG. 2. An exemplary device 100 is shown schematically in place in the entrance to the left coronary artery LCA in FIG. 3 (which is an enlargement of the highlighted portion of FIG. 2), and is further illustrated schematically in FIG. 4. The LCA is selected for purposes of illustration only—the device may be disposed in any part of the coronary tree.

Device 100 is configured to be placed or otherwise implanted or located in or near the entrance to a coronary artery, such as left coronary artery LCA. In this location, it would be upstream of atherosclerotic plaque in the coronary artery. The device is configured to allow passage of blood through or past the device, i.e. the device does not occlude the artery. The structure of device 100 is preferably biodegradable, so that the device eventually is absent from the ostium or artery. This can allow subsequent placement of other such devices as needed and/or passage of other devices into the coronary artery. Alternatively, the device structure may be partially or completely non-biodegradable and thus permanent.

Device 100 includes a body 110 providing structure for the device, and a reservoir 140 for a suitable therapeutic agent TA. The device is configured and/or formulated to release therapeutic agent into blood flowing through the coronary artery, so that the agent can be carried downstream to the site(s) of vulnerable plaque. The therapeutic agent TA is released over a desired time in a desired amount to provide a therapeutically effective amount of the therapeutic agent at the downstream location(s) of the vulnerable plaque(s).

The reservoir functionality may be achieved by incorporating the therapeutic agent into the constituent material of the device, and/or an additional layer or body of other material, to be eluted from the material (whether or not as part of biodegradation of the material). Alternatively, or additionally, the reservoir functionality may be achieved by forming a cavity in the device from which the agent can be released and/or by any other suitable mechanism.

Device 100 also includes a retention structure or mechanism 120, by which device 100 can be maintained in the desired location. The retention mechanism may be implemented as a mechanical fastener (such as a prong, spike, hook), or may be achieved through adhesion (forming the device of, or coating it with, a sticky/tacky material, and/or applying an adhesive). The retention mechanism may also implemented simply by the geometry of the device, conforming the device more or less closely to the shape of the ostium or artery wall and relying on friction and/or the pressure gradient of the blood flowing through the artery and/or the device to hold the device in place.

Device 100 may be formed externally to the body and then inserted into the desired location with conventional techniques (such as by a balloon catheter), or may be assembled and/or formed in situ.

Several exemplary implementations of device 100 are described in more detail below. The structure/geometry of the devices is described first, then the materials/compositions of the devices. This is followed by a description of suitable therapeutic agents.

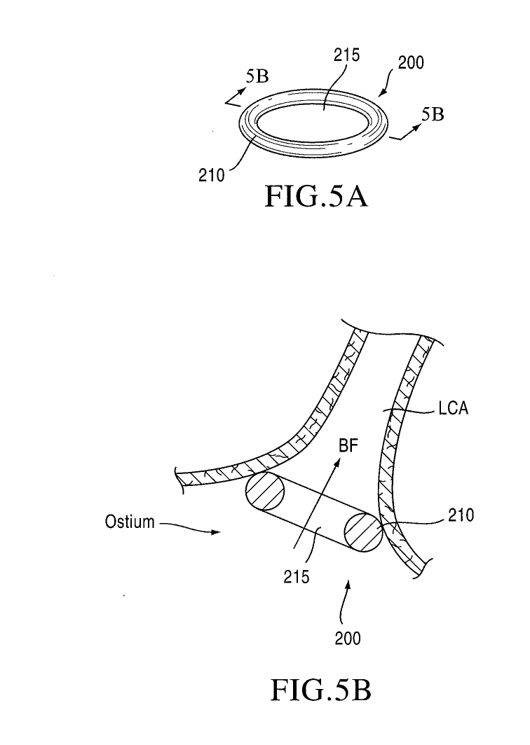

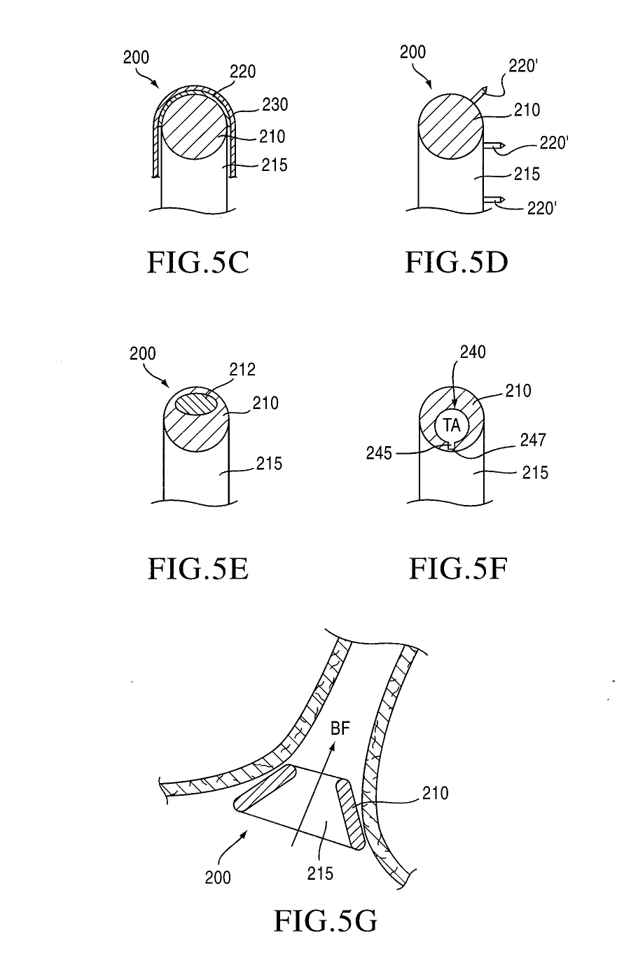

A first exemplary implementation, device 200, is illustrated in FIGS. 5A and 5B. In this embodiment, device 200 is configured as an annular ring, or torus. The body 210 of device 200 defines a lumen or aperture 215. When device 200 is in place in the artery (the LCA, as shown in FIG. 5B), blood flow BF in the artery can flow through aperture 215. Therapeutic agent TA released from body 210 can thus enter blood flow BF.

Device 200 may be delivered to the desired site in the coronary tree by any one of many techniques known to the artisan. For example, a catheter may be used for percutaneous translumenal delivery, such as the type used for delivering devices such as stents to the coronary tree. The device may be disposed over the balloon of a balloon catheter, and when the device is appropriately positioned, the balloon can be expanded to deliver the device. The device may also be self-expanding, and delivered using a guide catheter and guide wire.

Device 200 includes attachment mechanism 220. Several embodiments of attachment mechanism 220 are contemplated. In a first embodiment, shown in partial cross-sectional view in FIG. 5C, attachment mechanism 220 is implemented as a layer of adhesive. Deployment of the device 200 in the artery with the surface of the adhesive layer in contact with the artery will cause the adhesive to adhere to the wall of the artery and retain the device 200 in place. Rather than a separate layer of adhesive material, the material of which the body 210 of device 200 is formed may be sufficiently tacky or adhesive to provide the desired degree of adhesion. As noted above, the device may be delivered by a balloon catheter, and expansion of the balloon may urge the device into adhering contact with the artery wall. Alternately, the device may be self expanding, and when released from compressive constraint, may resiliently urge itself against the artery wall.

The adhesive surface of the ring (whether or not a separate layer of adhesive material) may be protected prior to delivery to the desired site in the coronary branch by a protective sleeve (sleeve 230, shown in FIG. 5C) that splits apart upon deployment of the device, exposing the adhesive surface for contact with the artery wall.

In an alternative embodiment, shown in partial cross-sectional view in FIG. 5D, attachment mechanism 220′ includes one or more mechanical fasteners in the form of a spike, hook, or pin that can be embedded into the artery wall to retain the device. Multiple fasteners may be arrayed about the perimeter of the device. The fasteners may be moveable between a stowed position (not shown) and the deployed position shown in FIG. 5D so that they do not protrude from the device until the device is delivered. They may be urged into the deployed position by expansion of the delivery balloon, or may be released from a frangible protective sleeve that is split by expansion of the delivery balloon. Other techniques, such as forming the fasteners from shape memory materials, will also be apparent to the artisan.

Rather than being formed as a torus, device 200 may take another annular shape, with a conical or other tapered outside surface than can engage the tapered artery wall, as shown in FIG. 5G. The device 200 is thus retained in the artery (and in particular prevented from slipping distally into the coronary tree) by the engagement of the matching tapers.

In each of the embodiments of device 200, above, the device may be formed of biodegradable, bioabsorbable, and/or bioerodable material(s). Such materials may include modified starches, gelatins, cellulose, collagen, fibrin, fibrinogen, connective proteins or natural materials (e.g., elastin), polymers or copolymers (e.g., polylactide [poly-L-lactide (PLLA), poly-D-lactide (PDLA), poly(lactic-co-glycolic acid) (PLGA)], polyglycolide, polydioxanone, polycaprolactone, polygluconate, polylactic acid (PLA), polylactic acid-polyethylene oxide copolymers, poly(hydroxybutyrate), polyanhydride, polyphosphoester, poly(amino acids), e.g. poly-tyrosine, poly(alpha-hydroxy acid)) or related copolymers of these materials, as well as composites and combinations thereof and combinations of other such materials.

Any of the above embodiments may be formed partly or wholly of materials that do not biodegrade, bioabsorb, and/or bioerode. For example, as shown in FIG. 5E, device 200 has a body 210 that includes a core or scaffold 212 formed of a material that is biocompatible but that does not biodegrade, bioabsorb, or bioerode. Thus, the other portion of body 210 will eventually be absent from the artery, but the scaffold 212 will remain. This design can help to avoid fragmentation of the body 210 as it degrades, by providing a structural support for the biodegradable component. Suitable materials for the scaffold 212 include polymer, metal, metal alloy (e.g., stainless steel, Ni/Ti alloy), or a combination or other suitable material. Scaffold 212 may be formed from autogenous/autologous, and/or synthetic biocompatible materials. Synthetic biocompatible materials may include silicone, rubber, polyurethane, polytetrafluoroethylene (PTFE), expanded polytetrafluoroethylene (ePTFE), polyester, Dacron, Mylar, polyethylene, PET (Polyethylene terephthalate), polyamide, polyamide, PVC, Kevlar (polyaramid), polyetheretherketone (PEEK), polypropylene, polyisoprene, polyolefin, or a composite of these or other suitable materials.

As discussed above, the reservoir functionality of the device may be achieved by incorporating the therapeutic agent into the biodegradable material of the device body, such as by formulating the therapeutic agent with a biodegradable polymer to a desired kinetic delivery rate (“KDR”). Alternatively, or additionally, the device could include a cavity within which a suitable quantity of therapeutic agent is stored and can be released through an opening in the cavity. Such an embodiment is illustrated schematically in FIG. 5F. Cavity 240 is filled with therapeutic agent TA, and communicates with aperture 215 via opening 245. Opening 245 may optionally be closed by a biodegradable closure or plug 247, so that therapeutic agent TA cannot be released from device 200 until after the device has been deployed in the artery and the plug 247 biodegrades by exposure to blood.

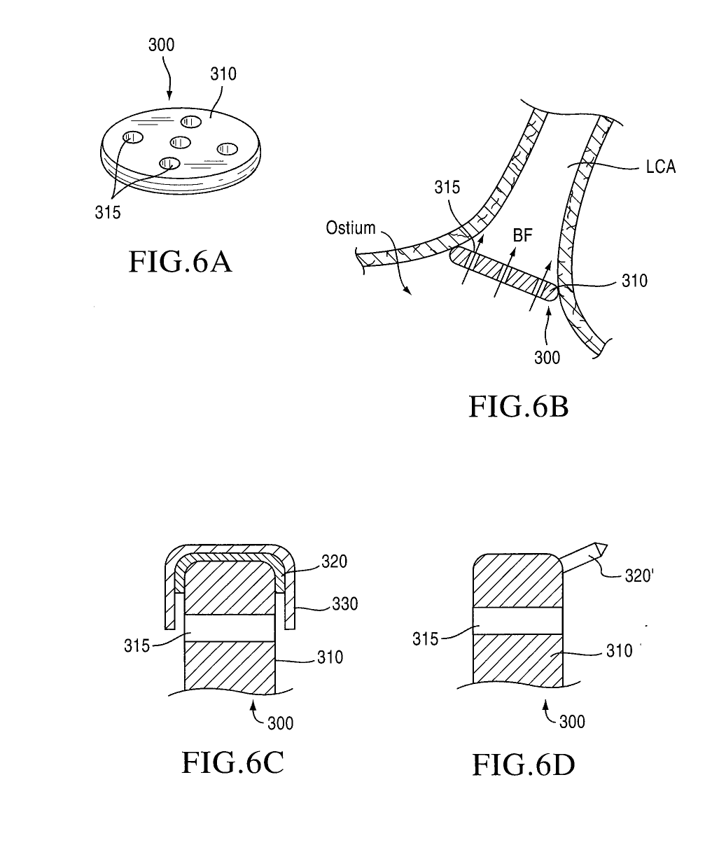

A second exemplary implementation, device 300, is illustrated in FIGS. 6A and 6B. This embodiment is similar to the prior embodiment except that body 310 is configured as a disk that is perforated with multiple lumens or apertures 315. When device 300 is in place in the artery (as shown in FIG. 6B), blood flow BF in the artery can flow through apertures 315. Therapeutic agent TA released from body 310 can thus enter blood flow BF. Device 300 can also include retention mechanism 320, with implementations similar to those for device 200. Thus, as shown in FIG. 6C, attachment mechanism 320 may be implemented as a layer of adhesive (or the material of which the body 310 of device 300 is formed may have the desired adhesive properties). Similarly, the adhesive surface of the ring may be protected prior to delivery to the desired site in the coronary branch by a protective sleeve 330. Alternatively, as shown in FIG. 6D, attachment mechanism 320′ includes one or more mechanical fasteners, with the same options and variations as described for device 200.

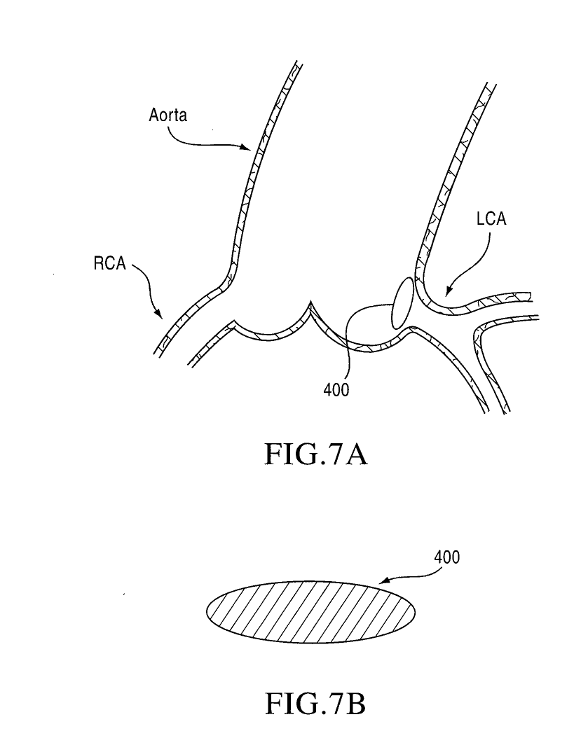

In a further embodiment, device 400 is similar to device 300, but is formed as a mesh that can be disposed across the ostium, as shown in FIGS. 7A and 7B.

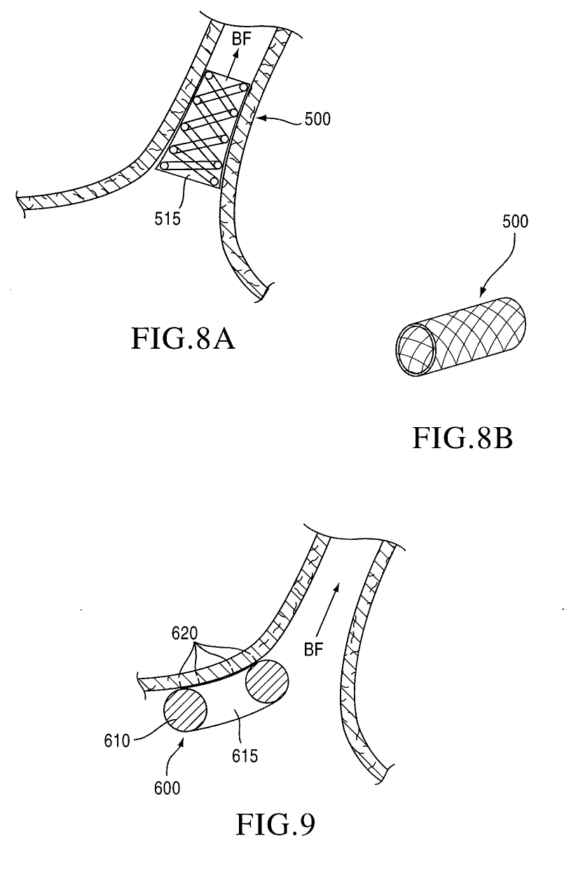

In yet a further embodiment, device 500 is formed similarly to known drug-eluting coronary artery stents. As shown in FIGS. 8A and 8B, device 500 may be a tubular mesh stent with a central lumen 515. The stent may be deployed/expanded by a balloon, or may be self expanding. When deployed, blood flow BF can pass through lumen 515.

The device need not be disposed around the interior perimeter of the artery, or entirely across the ostium, as with the embodiments described above. It is sufficient that the device can release therapeutic agent TA into the blood flow entering the coronary branch. Thus, for example, as shown in FIG. 9 a device 600, similar to device 200, may be anchored to the wall of the coronary artery, or to the aorta, such as by an attachment mechanism 620 with mechanical fasteners penetrating the artery or aorta wall. Therapeutic agent TA can be released from device 600 and enter blood flow BF, even though no blood flows through aperture 615.

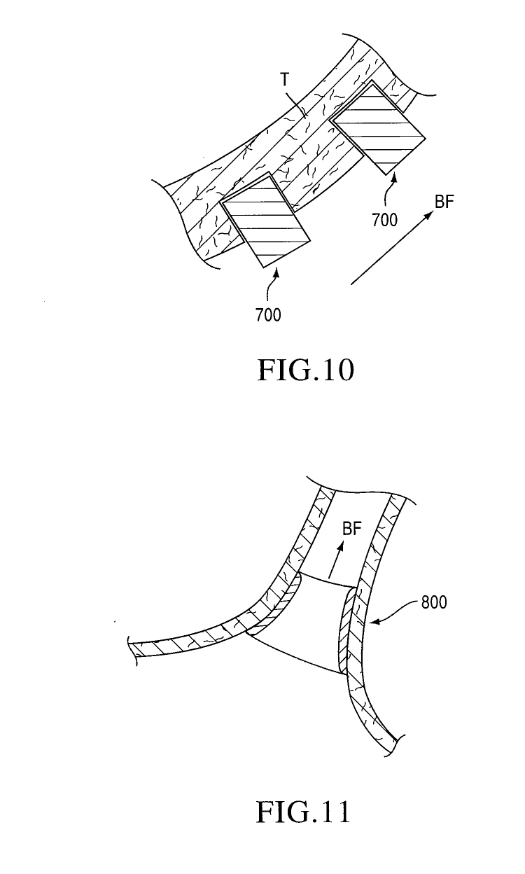

The artisan will recognize that there can be many suitable shapes and geometries for device 600, including a flat sheet or plate, a disk, etc. Further, the device need not be disposed in the lumen or the aorta or artery and attached to the wall. Rather, the device may be partially or wholly embedded into the vessel wall. Thus, as shown in FIG. 10, one or more devices 700 may be formed as plugs that can be implanted into the tissue T around or inside the ostium. Therapeutic agent released from device(s) 700 enters blood flow BF into the coronary tree. In the embodiments described above, the device is fabricated or assembled externally to the body, and is then delivered in complete form to the desired location in the body. In other embodiments, the device may be formed or assembled in situ. For example, as shown in FIG. 11, device 800 may be formed directly on the artery wall as a layer of, for example, polymer material that can be delivered in uncured (e.g. liquid) form and cured in place. The artery is thus endoluenally paved with drug-eluting material. Device 800 can be formed to a desired thickness and axial and peripheral extent. It may adhere to the artery wall, or may simply be held in place its mating fit with the shape of the artery. The polymer may be delivered to the desired location by a porous balloon or a catheter, as will be apparent to the artisan.

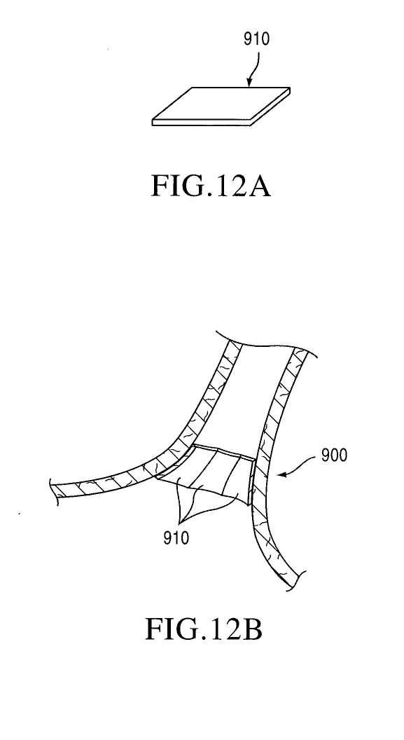

As a further alternative, a device 900 may be formed or assembled in place from preformed sheets of material, rather than from a liquid polymer. Thus, as shown in FIGS. 12A and 12B, device 900 may be formed from multiple sheets 910 of suitable material. The sheets can then be delivered to the desired location and laid onto the vessel wall, abutting or overlapping.

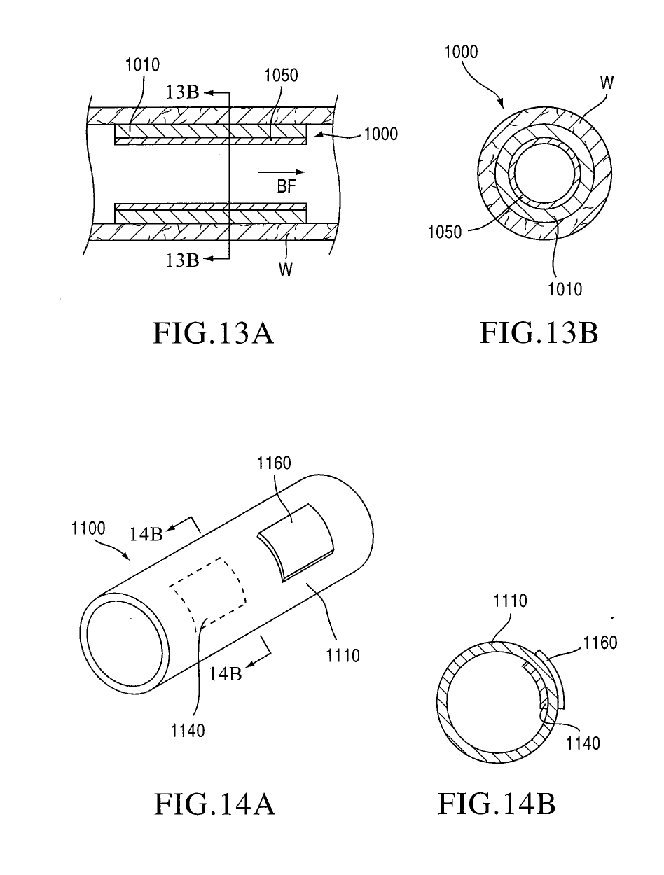

In the embodiments above, the therapeutic agent TA can be released from the device immediately upon placement of the device and ensuing exposure to blood flow BF. The therapeutic agent is then continually released in amount as a function of time that can be tailored through a variety of factors, including the geometry and composition of the device and the therapeutic agent. It may be preferred to delay the onset of release of the therapeutic agent, which may correspondingly mean delaying the onset of biodegradation of the body of the device. This can be accomplished with an absorption inhibitor layer on the body of the device. This is shown schematically in FIGS. 13A and 13B for a device 1000 with a body 1010 (which may be similar to the cylindrical stent-like embodiment of device 500, above) that is placed in an artery adjacent the artery wall W, with blood flow BF passing through the device. The absorption inhibitor layer 1050 can reduce the rate of absorption of the device body 1010 that it overlies, and may reduce the rate of absorption to zero. If the absorption inhibitor layer 1050 itself is absorbed, its effect on the rate of absorption of the underlying device body 1010 is eliminated once the absorption inhibitor layer 1050 is completely absorbed. If the absorption inhibitor layer 1050 is not absorbed, its effect persists until the underlying device body 1010 is absorbed (through the absorption inhibitor layer 1050 at a reduced rate and/or from another direction). Thus, the duration of the absorption inhibitor layer's effect on the rate of absorption of the underlying device body 1010 depends on the rate of absorption of the absorption inhibitor layer 1050 and/or its thickness.

By varying the thickness of the absorption inhibitor layer 1050 on a particular portion of device body 1010, the absorption of some portions of the device body 1010 can be delayed longer than other portions. Portions of the device body 1010 may have no absorption inhibitor layer 1050. These portions of device body 1010 will begin to biodegrade, and release therapeutic agent TA, immediately upon implantation into the body lumen. Alternatively, the thickness of the absorption inhibitor layer 1050 may be constant. This approach to inhibiting bioabsorption/biodegradation of an endolumenal device is described in more detail in copending, commonly-assigned application Ser. No. 11/213,817, filed Aug. 30, 2005, the disclosure of which is incorporated herein by reference.

As an alternative to the delayed release of the therapeutic agent described above, it may be desirable to more selectively or episodically release the therapeutic agent into the coronary tree. Several techniques are contemplated for achieving this goal. For example, the therapeutic agent may be delivered systemically when needed, but in a form that requires activation to be effective (and correspondingly to have any undesired side-effects on parts of the circulatory system other than the coronary tree), which may be referred to as a prodrug. The prodrug could then be activated locally in the coronary tree by, for example, an externally-applied energy source such as [electromagnetic radiation of various frequencies (RF, microwave, High-Intensity Focused Ultrasound (HIFU)). The activation could be enhanced, and/or further localized, by placing a device in the ostium or coronary artery entrance, as with the devices above, to serve as an antenna for focusing the externally-applied energy and activate the prodrug as it passes by the device and into the coronary tree.

In an alternative embodiment, the therapeutic agent may be contained within a device such as those disclosed above, rather than being introduced systemically, and can be selectively released from the device by external activation, such as by an external energy source. In this embodiment, shown schematically in FIGS. 14A and 14B, device 1100 has a body 1110 and a reservoir 1140 of therapeutic agent TA. A vibration device 1160 is coupled to the body 1110, and is configured to cause movement of the body such that at least a portion of the therapeutic agent TA is released from the reservoir 1140.

The vibration device 1160 can be, for example, an oscillator, such as a micro-oscillator, that is coupled to the body 1110. The vibration device 1160 can vibrate the body 1110 at a resonance frequency associated with the particular configuration of device 1100. Upon activation, the vibration device 1160 can vibrate the body 1110 such that the therapeutic agent TA is released from the reservoir 1140 at a rate different from a rate of release associated with the therapeutic agent TA without the body 1110 being vibrated. This approach to controlling the release of a therapeutic agent from a medical device is described in more detail in copending, commonly-assigned application S/N [to be included when available)], filed Apr. 6, 2006, the disclosure of which is incorporated herein by reference.

The devices described above may be used in methods of treating atherosclerotic plaques in a coronary artery, such as of a mammal. Such method(s) include disposing at or proximate to the entrance of the coronary artery a device as described above configured to be retained in or proximate to the entrance to the coronary artery and formulated to at least partially biodegrade by exposure to blood passing through the coronary artery; and releasing from the device into the blood a therapeutic agent for the treatment of the vulnerable plaque. The therapeutic agent is releasable from the device into blood passing across one or more surfaces of the device and is transportable by the blood to the plaque in a therapeutic amount. The device may be disposed in one of the ostium, the left coronary artery, or the right coronary artery, or elsewhere in the coronary tree of the subject, such as a mammal.

The devices of the various embodiments described above may be formed of various materials and with various constructions. As noted above, the device may be wholly or partly biodegradable. The body and other structure elements of the devices may be preshaped from biocompatible materials that substantially inhibit deformation of the structural element. An externally placed structural element may be formed from one solid continuous piece of biocompatible material, or may be formed from more than one type of material. The structural element may be fabricated using various methods and processes including sintering, molding (e.g., injection molding), casting, adhesive bonding, laminating, dip coating, spraying as well as composites and combinations thereof and combinations of other suitable methods and processes.

The device can be permanently placed in or near the artery, or may be placed in the vessel for a desired time and then removed. Optionally, a device that is removed, or that completely biodegrades, may be replaced with a similar device.

A structural element may be partially or completely fabricated from materials that swell or expand when they are exposed to a fluid (e.g., blood, another body fluid, or an infused fluid). These materials may include hydrophilic gels (hydrogels), foams, gelatins, regenerated cellulose, polyethylene vinyl acetate (PEVA), as well as composites and combinations thereof and combinations of other biocompatible swellable or expandable materials.

A structural element may include a surface coating. The surface coating may be formed from biocompatible materials. Applying a biocompatible surface coating to the structural element may allow the structural element to be formed from one or more potentially non-biocompatible materials.

At least one coating may be located on a surface, as well as inside a structural element. The structural element may be coated with hydrophilic materials that are biologically inert. The element may incorporate one or more coatings, materials, compounds, substances, drugs, therapeutic agents, etc. that treat vulnerable plaques. In some embodiments, a structural element may be formed from multiple layers.

### Therapeutic Agents

A variety of therapeutic agents are contemplated for use in the method, and with the apparatus, of the disclosed inventions. These include, but not limited to, anti-inflammatory agents, metalloprotease inhibitors, sclerotic agents (to stabilize “thicken” the thin cap fibrous atheroma of the vulnerable atherosclerotic plaque) and anti-lipid agents.

As discussed above, the breakdown of connective tissues, including proteoglycan and collagen, leading to resorption of the extracellular matrix is a feature of many pathological conditions, such as rheumatoid and osteoarthritis, corneal, epidermal or gastric ulceration, tumor metastasis or invasion, periodontal disease, bone disease and atherogenesis. There is a body of evidence that show that matrix metalloproteases (MMPs) are important in the uncontrolled breakdown of connective tissue, including proteoglycan and collagen, leading to resorption of the extracellular matrix. Normally MMPs are tightly regulated at the level of their synthesis as well as at their level of extracellular activity.

Atherogenesis involves two key events: migration of circulating monocytes and other inflammatory cells into the subendotherlium and migration of smooth muscle cells from the media to intima. Eventually, plaque erosion and rupture may directly precipitate thrombosis and eventually damage to the heart. These processes share a common requirement, focal matrix degradation, which is predominantly accomplished by the proteolytic action of locally expressed and activated MMPs. A variety of extracellular stimuli, including cytokines, cell-to-cell, and cell-to-matrix interactions can induce MMP expression. Of particular relevance to atherosclerotic pathology is an increase of expression and activity of MMPs have been noted in vulnerable plaques regions (Galis et al. (1994) J. Clin. Invest., 94, 2493-2503).

Thus, compounds that inhibit metalloprotease activity are of therapeutic importance for the treatment of inflammatory disorders, including vulnerable plaques. Accordingly, it is contemplated that at least one metalloprotease inhibitor or pharmaceutically acceptable salts or prodrugs thereof may be released high in the coronary arterial tree for treatment of downstream vulnerable plaques. Similarly, it is contemplated that a combination of therapeutic agents comprising an MMP inhibitor, a pharmaceutically acceptable salts or prodrugs thereof may be released high in the coronary arterial tree for treatment of downstream vulnerable plaques. Exemplary, non limiting, examples of metalloprotease inhibitors are magnesium gluconate, Sopar, Pharmaprojects No. 3813, Pharmaprojects No. 5682, batimastat, matrix metalloproteinase inhibitors—3-Dimensional Pharmaceuticals, BAY 157496, TIMP-3 gene therapy, metalloproteinase inhibitors—OSI/Vernalis, PG 116800, PGE 5747401, metalloenzyme inhibitors form Serono/Vernalis, CH 715, TIMP-4 gene therapy, COL 3, Pentosan polysulfate, Ursolic acid, LY 290181, REGA 3G12, matrix metalloproteinase inhibitors from Cengent Therapeutics/De Novo, MMP inhibitors from Millennium, rebimastat, RO 1130830, apratastat, and ABT 518.

Since evidence suggests that inflammation plays a central role in the cascade of events that results in vulnerable plaque formation, anti-inflammatory agents and immunomodulatory agents are also contemplated as therapeutic drugs usable with the apparatuses and methods of the disclosed inventions. Anti-inflammatory agents and immunomodulatory agents can be delivered independently, concurrently, or in combination with any therapeutic drug. Examples of anti-inflammatory agents are: adrenocorticoids, corticosteroids (e.g., beclomethasone, budesonide, flunisolide, fluticasone, triamcinolone, methlyprednisolone, prednisolone, prednisone, hydrocortisone), glucocorticoids, steroids, non-steriodal anti-inflammatory drugs (e.g., aspirin, ibuprofen, diclofenac, and COX-2 inhibitors), leukotreine antagonists (e.g., montelukast, methyl xanthines, zafirlukast, and zileuton), β2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines. Any anti-inflammatory agent, including agents useful in therapies for inflammatory disorders, well-known to one of skill in the art can be used. Non-limiting examples of anti-inflammatory agents include non-steroidal anti-inflammatory drugs (NSAIDs), steroidal anti-inflammatory drugs, anticholinergics (e.g., atropine sulfate, atropine methylnitrate, and ipratropium bromide.

It is also contemplated that anti-proliferative agents can be used with the apparatuses and methods of the disclosed inventions. Exemplary anti-proliferative agents include paclitaxel, Alkeran, Cytoxan, Leukeran, Cis-platinum, BiCNU, Adriamycin, Doxorubicin, Cerubidine, Idamycin, Mithracin, Mutamycin, Fluorouracil, Methotrexate, Thoguanine, Toxotere, Etoposide, Vincristine, Irinotecan, Hycamptin, Matulane, Vumon, Hexalin, Hydroxyurea, Gemzar, Oncovin, Etophophos, tacrolimus (FK506), Everolimus, or any of the following analogs of sirolimus: SDZ-RAD, CCI-779, 7-epi-rapamycin, 7-thiomethylrapamycin, 7-epi-trimethoxyphenyl-rapamycin, 7-epi-thiomethylrapamycin, 7-demethoxy-rapamycin, 32-demethoxy, 2-desmethyl and proline. Other suitable anti-proliferative agents will be apparent to the artisan.

It is further contemplated that lipid lowering agents and/or statins can be used with the apparatuses and methods of the disclosed inventions, singly or in combination thereof, to influence the composition of the lipid pool in the vulnerable plaque. Any lipid-lowering agent well-known to one of skill in the art can be used in the compositions and methods of the invention. Non-limiting examples include lovastatin, pravastatin, atorvastatin, and cerivastatin.

It is further contemplated that anti-thrombogenic agents can be used with the apparatuses and methods of the disclosed inventions, singly or in combination thereof. Non-limiting examples of anti-thrombogenic agents include heparin or coumadin, or anti-platelet agents, such as Plavix or ReoPro.

The therapeutic agents listed above are not an exhaustive list, but rather are just examples of the types of therapeutic agents that can be used with the apparatuses and methods of the disclosed inventions. In addition, combination therapy with the above listed drugs or any other therapeutic agents are also contemplated.

The device may be configured and formulated to deliver treatment regime(s) gradually over time, e.g. 1 to 6 months, 6 to 12 months, 12 to 24 months or longer if desired. A person of skill in the art can configure and formulate the device to deliver therapeutic drugs at a desired rate. In addition, the device can be configured and formulated to elute therapeutic drugs simultaneously or consecutively. Thus, the device may elute one therapeutic agent for a length of time and then elute another therapeutic agent after the first has been eluted. Alternatively, the therapeutic agents may be released simultaneously.

It will be appreciated by persons skilled in the art that numerous variations and/or modifications may be made to the embodiments without departing from the spirit or scope of the claims as broadly described. Equivalents for the particular embodiments discussed in this description may practice the claims as well. The present embodiments are, therefore, to be considered in all respects as illustrative and not restrictive.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present application before the priority date of each claim of this application.

What is claimed is:

1. Apparatus comprising:
a body configured for placement at or proximate to an entrance to a coronary artery and upstream of an atherosclerotic plaque in the coronary artery, said body being formulated to biodegrade; and
an effective amount of a therapeutic agent for the treatment of the atherosclerotic plaque, said therapeutic agent being releasable from said body into blood passing across a surface of said apparatus.

2. The apparatus of claim 1, wherein said therapeutic agent is selected from the group consisting of anti-inflammatory and anti-lipid agents.

3. The apparatus of claim 2, wherein said body is configured for placement in one or more of the ostium, the left coronary artery, and the right coronary artery of a mammal.

4. The apparatus of claim 1, wherein said body is configured as an annular ring.

5. The apparatus of claim 1, wherein said body is composed of a biodegradable polymer.

6. The apparatus of claim 5, wherein said therapeutic agent is contained in said biodegradable polymer and is formulated to be eluted from said body upon biodegradation of said biodegradable polymer.

7. The apparatus of claim 1, wherein said body is configured and formulated to be substantially completely biodegraded in a mammalian body within 6 to 12 months.

8. The apparatus of claim 1, wherein said body is configured with a taper that approximates the taper of the coronary artery.

9. The apparatus of claim 1, further including means for anchoring said body at or proximate to the entrance to the coronary artery.

10. The apparatus of claim 1, wherein said body is formulated to adhere to an inner surface of a mammalian blood vessel.

11. The apparatus of claim 10, wherein said body is formed of a tacky polymer.

12. The apparatus of claim 1, wherein said body includes at least one projection configured to be embedded into a wall of a mammalian blood vessel to anchor said body to the blood vessel.

13. The apparatus of claim 1, wherein said body is resiliently expandable from a compressed configuration to a larger, deployed configuration and wherein said body is configured for placement in a coronary artery such that the body can be anchored in the coronary artery by resilient expansion toward said deployed configuration.

14. The apparatus of claim 1, wherein said body includes a first portion that is substantially non-biodegradable and a second portion that is biodegradable in a mammalian blood vessel.

15. A method comprising:
disposing at or proximate to the entrance of a coronary artery a device configured to be retained in the coronary artery and formulated to at least partially biodegrade by exposure to blood passing through the coronary artery and to release into blood passing through the coronary artery for delivery to a vulnerable plaque in the coronary artery downstream of said device a therapeutic amount of a therapeutic agent for the treatment of the vulnerable plaque.

16. The method of claim 15, wherein said device is an annular ring having a central opening and wherein said disposing includes disposing said annular ring so that blood flowing through the coronary artery flows through said opening.

17. The method of claim 15, wherein said disposing includes disposing said device in one of the ostium, the left coronary artery, or the right coronary artery of a mammal.

18. The method of claim 15, wherein said device includes a layer containing said therapeutic agent.

19. The method of claim 18, wherein said layer is formed of a biodegradable polymer.

20. The method of claim 19, wherein said device is formulated to release said therapeutic agent upon biodegradation of said polymer.

21. The method of claim 15, wherein said device is configured with a taper that approximates the taper of a portion of the coronary artery and wherein said disposing includes disposing said body so that said taper is engaged with the taper of the portion of the coronary artery.

22. The method of claim 15, wherein said disposing includes adhering said device to an inner surface of the coronary artery.

23. The method of claim 22, wherein said body includes a tacky polymer disposed on a least a portion thereof and wherein said disposing includes engaging said tacky polymer with the inner surface of the coronary artery.

24. The method of claim 15, wherein said device includes a projecting portion and further comprising anchoring said device in the coronary artery by embedding said projecting portion into an inner surface of the coronary artery.

25. The method of claim 15, wherein said disposing includes delivering said device translumenally.

26. The method of claim 17, wherein said device is resiliently expandable from a compressed configuration to a larger, deployed configuration and wherein said disposing includes delivering said device in said compressed configuration to the coronary artery and allowing said device to resiliently expand toward said deployed configuration and to engage an inner wall of the coronary artery.
